(19) United States
(12) Patent Application Publication
(10) Pub. No.: US 2007/0172529 A1
(43) Pub. Date: Jul. 26, 2007

(54) Herbal formulation for the treatment of piles

(75) Inventors: Pushpangadan Palpu, Lucknow (IN); Aiay Kumar Singh Rawat, Lucknow (IN); Chandana Venkateswara Rao, Lucknow (IN); Sharad Kumar Srivastava, Lucknow (IN); Ramaswami Kartik, Lucknow (IN)

Correspondence Address: LADAS & PARRY, 26 WEST 61ST STREET, NEW YORK, NY, 10023, US

(73) Assignee: COUNCIL OF SCIENTIFIC AND INDUSTRIAL RESEARCH

(21) Appl. No.: 11/651,410

(22) Filed: Jan. 8, 2007

Related U.S. Application Data
(63) Continuation of application No. 11/024,019, filed on Dec. 28, 2004, now abandoned.

(51) Int. Cl. A61K 36/48

(52) U.S. Cl. 424/725; 424/757; 424/779

(57) ABSTRACT

The present invention provides a novel herbal formulation for use in treatment of piles. Formulation(s) comprises of leaves of Moringa oliefera, tender leaves of Pongaemia pinnata, whole plant of Cassia occidentalis and leaves of Albizia lebbeck. The formulation can be used as an emulsion or as a soft gelatin capsule for oral dosage forms or in the form of ointment. Moringa olifera used as carminative, stomachic and abortifacient. Pongaemia pinnata is useful in treatment of diseases like dairrhoea, flatulence and for cleaning ulcer and sores. Cassia occidentalis is used for treatment of hysteria, dysentery and other stomach problem. Albizia lebbeck is used in inflammation, itching, leucoderma and also used for strengthening of gums.

### FIELD OF THE INVENTION

The present invention relates to a novel herbal formulation useful in the treatment of piles.

### BACKGROUND AND PRIOR ART OF THE INVENTION

A major discovery of the past two decades in the field of gastrointestinal disorder has been the elucidation of understanding the mechanism of anorectal problem. This is commonly known as piles or haemorrhoids. The word haemorrhoids is derived from Greek—haima meaning blood and rhoos meaning flowing. Piles is derived from Latin-pila meaning a ball. Haemorrhoids are veins occurring in relation to the anus. Such haemorrhoids may be external or internal-external or internal to the anal orifice. The external variety is covered by the skin, while the internal variety lie beneath the anal mucous membrane. When the two varieties are associated, they are known as intero-external haemorrhoids. Varicosities in veins are regions that are enlarged and inflamed. In the rectal veins, varicosities are known as haemorrhoids (piles). Haemorrhoids develop when the veins are put under pressure and become engorged with blood. If the pressure continues, the wall of veins stretches. Such a distended vessel oozes blood, and bleeding or itching are usually the first signs that a haemorrhoids has. developed. Stretching of a vein also favours clot formation, further aggravating swelling and pain. Haemorrhoids may be caused by constipation, which may be brought on by low-fiber diets (Gaj et al; Chir Ital, 56, 699; 2004). Also, repeated straining during defecation forces blood down into the rectal veins, increasing pressure in these veins and possibly causing haemorrhoids. Piles, or haemorrhoids, are areas in the anal canal where the tissue, which contains lots of blood vessels, has become swollen. They can be internal, occurring inside the anus, or external, when they can be seen and felt on the outside of the anus. When visible they look like round pink swellings, the size of a pea or a grape. By the age of 50, up to half the population of the UK will have suffered from piles at some point. Piles are common in pregnant women, but are rare in children. The veins, which form internal haemorrhoids, become engorged as the anal lining descends and is gripped by the anal sphincters. The mucosal lining is gathered prominently in three places (the ‘anal cushions’), which can be in the areas of the three terminal branches of the superior haemorrhoidal artery, but this is exceptional. The anal cushions are present in embryonic life and are necessary for full continence. Straining causes these cushions to slide downwards, and internal haemorrhoids develop in the prolapsing tissues (Basdanis et al; Sug Endosc, 2; 2004). The exact way piles form is controversial but it may be linked to excess pressure in the anus and lower rectum. This pressure can have one or more of several different causes like Straining to empty the bowels when constipated, Chronic diarrhoea, Pregnancy—the weight of the foetus on the abdomen and the increased blood flow, as well as the effect of hormones on the blood vessels, Childbirth—pushing during childbirth increases the pressure in the veins, Straining to pass urine, especially in men with prostate problems, Cancer or growths in the pelvis or bowel, which may exert pressure in a similar way to a pregnancy, Family history—piles can run in families and are potentially hereditary, perhaps because of weak veins in the anal area, Obesity, Varicose veins—many people with these also develop piles, although piles are not varicose veins. There are different types of piles, which are classified according to their position firstly; First-degree piles remain inside the rectum or anal canal. Second-degree piles protrude (or prolapse) from the anus when the bowels are opened, but return of their own accord afterwards. Third-degree piles are similar, but only return inside when pushed back. Fourth-degree piles hang permanently outside the anus needs surgical intervention. Most people affected have internal piles and may not have any symptoms at all. The earliest symptom is often bleeding of fresh, red blood from the anal passage when the bowels are opened. There may be itchiness around the anal area. Third and fourth degree piles may be more painful and tend to produce a slimy discharge of mucus that leaks from the exposed lining of the pile. There is possible complication, which includes factors like Strangulation, which occurs when a prolapsed pile swells considerably and the blood flowing into it cannot return to the body. This is very painful and can lead to thrombosis. Another factors are thrombosis, when the bloods in the swollen pile clots. A thrombosed pile may be less painful than a strangulated pile and looks different, being dark purple or black in colour. One more factors are Gangrene where there is lack of blood supply to a pile may lead to severe pain and gangrene (tissue death). This is a dangerous and rare complication needing immediate surgery. One more factor is Infection; this is a rare but serious complication. An abscess may form around the rectum and anus causing pain and swelling. Rarely, the infection may spread, particularly to the liver. This is because the blood draining away from the anal area passes through the liver. Ultimately the last one is anaemia where Severe loss of blood from piles. Bleeding over a long period of time can cause anaemia, when there are not enough blood cells to supply the body with oxygen.

Relief can be obtained depending on different factors. For external piles that cause irritation may be helped by application of soothing creams, which are available over-the-counter from pharmacies. These creams lubricate the area and some contain a local anaesthetic to provide short-term relief from any discomfort. Regular warm baths may relieve the irritation. However, over-use of soaps is not recommended since it may increase irritation. Ice packs may help reduce swelling, but should not be applied directly to the skin. The next step, which is very essential, is treatment, which can be done by various methods Piles can usually be treated at home. The most important element in encouraging existing piles to clear up is to avoid constipation. By having regular bowel movements, and avoiding straining, stools pass easily and do not put pressure on the blood vessels in the anal area. Eating plenty of fibre-rich foods such as fruit, vegetables and wholegrain cereals (eg brown rice, wholemeal bread and wholemeal pasta), and drinking plenty of fluids, especially water, should keep bowel movements soft. It may help to take a fibre supplement such as isapghula husk (Fybogel) or mild laxatives such as lactulose solution (Duphalac), which soften bowel motions. Do not use strong laxatives, such as the stimulant laxative senna, on a routine basis unless advisable, because long-term use of such laxatives can be harmful. There are various methods in modern technology which are been used such as

Sclerotherapy is one method wherein the piles is injected with a chemical known as a sclerosant, which causes the piles to shrink. This is quick and relatively painless, but may have to be repeated once or twice. Banding, applying an elastic band above them treats the piles, so the blood supply to them is reduced. Traditional medicine is also known and is considered to be the most effective method of eliminating piles. Taking this we have come to the better outcome of treatment for piles by exploring a novel herbal treatment using different potent plant extracts. The role of medicinal plants in treatment of piles is much underestimated. Our studies show that herbal formulation(s) have the property of treating anorectal problem and can be used in treatment of piles as a formulation and act as a strong relieving factor in controlling piles.

### OBJECT OF THE INVENTION

The main object of the present invention is to provide a novel herbal formulation in dosage form useful in the treatment of piles.

Another objective of the present invention is to prepare herbal dosage form that treatment of constipation.

Yet another object of the present invention is to prepare herbal dosage from in the form of capsule for easy consumption or in the form of ointment or passeries.

### SUMMARY OF THE INVENTION

Accordingly, the present invention provides a novel herbal formulation(s) obtained from decoction of four potent plants and possessing the property of improving general health and used in treatment of diseases of blood, stomach problem and piles.

The novelty in the invention resides in a herbal formulation having the property of nerve relaxation and antinociceptive properties and its use as intoxicant, stomachic, anti-bacterial, expectorant, tonic, rheumatism, boil and swelling.

In one embodiment of the invention the herbal anti-hemorrhoids synergistic formulation (s) contains extracts of plants in pharmacologically effective form.

In another embodiment of the invention, the plants are selected from Pongaemia pinnata, Moringa olifera, Cassia occidentalis and Albizia lebbeck and are useful in the treatment of piles and hemorrhoids.

In yet another embodiment, the composition is synergistic mixture of plant extracts having anti-inflammatory, cooling, diuretic, nerves relaxant properties and antinociceptive properties.

In still another embodiment, the amounts of the plant extracts are as follows: Moringa olifera (2-4 Wt %), Pongaemia pinnata (3-4 Wt %), Cassia occidentalis (2-4 Wt %) and Albizia lebbeck (1-4 Wt %) the balance being conventional additives.

In another embodiment, the extract of Moringa olifera is obtained from leaves.

In another embodiment, the extract of Pongaemia pinnata is obtained from leaves.

In yet another embodiment, the extract of Cassia occidentalis is obtained from whole plant.

In yet another embodiment, the extract of Albizia lebbeck is obtained from bark.

In still another embodiment, the formulation also has the property of intoxicant and antibacterial, antifungal and used in treatment of diseases of blood, stomach problem and piles.

In yet another embodiment, the formulation is also used to ulcer and sores and in relieving rectal inflammation.

In another embodiment, the formulation is also used to cure wounds and fractures.

In yet another embodiment, the said formulation shrinks piles mass, control bleeding and hasten the healing process in inflamed skin and mucous membranes.

In still another embodiment, the formulation has an anti-inflammation and reduces constipation, which is one of the important criteria for being an effective measure against haemorrhoids.

In another embodiment, the formulation has pain relief and soothing action and facilitates the smooth evacuation of faeces.

In another embodiment, the formulation has laxative property which correct chronic constipation associated with haemorrhoids.

### DETAILED DESCRIPTION OF THE INVENTION

The present invention provides an herbal formulation useful in the treatment of piles. The tender leaves of Pongaemia pinnata, which are been used in treatment of diseases like dairrhoea, flatulence and for cleaning ulcer and sores. Leaves of Moringa olifera are used as carminative, stomachic and abortifacient. Cassia occidentalis are used for treatment of hysteria, dysentery and other stomach problem and Albizia lebbeck is used in inflammation, itching, leucoderma and also used for strengthening of gums. It was shown that the formulation produces a significant improvement in general health. The plants used in the present invention have the following properties.

Pongaemia pinnata Family: Papilionaceae

Botanical description: A widely distributed palaeotropical species common throughout India. A medium sized, semi-evergreen tree upto 18 m tall with a short bole and spreading crown; bark grayish-green or brown, smooth or covered with tubercles. Leaves alternate, imparipinnate, 5-9 foliate, dark green shining; leaflets opposite, ovate to elliptic, shortly acuminate, glabrous, to 15 cm long and 8 cm broad. Flower small, lilac or white tinged with pink or violet, fragrant, borne in fascicles of 2-4 flowers in axillary racemes to 15 cm long; calyx campanulate, truncate; corolla much exserted, standard suborbicular with curved folds above the claw, wings obliquely oblong. Fruit (pods) compressed, woody, glabrous, indehiscent, yellowish-grey when mature varying in size and shape, elliptic to obliquely oblong, to 7.5 cm long and 3.5 cm wide,with a curved beak, usually 1-seeded; seed elliptical or kidney shaped, to 2 cm long and 1.8 cm wide, wrinkled, reddish-brown. Depending on locality, flowering occurs between March and June; fruits mature between February and April.
Medicinal uses: The seed oil, whose active principle has been identified as karanjin, is highly valued in traditional Indian medicine. It is considered anthelmintic and styptic and is used externally in the treatment of chronic fevers, rheumatism herpes, scabies, leucoderma and other skin diseases. The seed oil posseses insecticidal, piscicidal and anti bacterial properties and is sometimes used as a lampoil in villages. The juice of leaves is used to treat flatulence, dyspepsia, diarrhoea and cough; it is considered a remedy for leprosy and gonorrhoea. A hot infusion of the leaves is used as a medicated bath to relieve rheumatic pains and for cleaning ulcers and sores. A paste of leaves is applied on the head to heal ulcer and cure dandruff in northeastern Karnataka. The flowers are considered a remedy for diabetes. The juice of the roots is also used for cleaning sores, as well as for cleaning teeth and strengthening gums; the stem bark is used to treat bleeding piles.
Phytochemistry: A polysaccrides consisting of arabinose (43.9), galactose (23.1), and glucuronic acid (23.5%) isolated from gum and partially characterized (Ind J Chem 1990,29b,545). Isolation of two new β-hydroxychalcones-ponganones I and II—from root bark and their characteristic (Chem. Pharm. Bull 1991,39,1473). New chromenoflavanone-(−)isoglabrachromene—and two new esters-hexaconsanyl caffeate and tri contanyl caffeate-isolated and their structure determined. Tetra-o-methylfisetin and ponga chromene isolated from roots and stem bark (Ind j chem., 1969,7,1275; Current science, 1973,42,128; Ind j chem., 1977,1513,12); a new furanoflavone-3-methoxypongapin- in addition to karanjan, kanjone and its two isomers 7-methoxy furano-(4,5,-6,5)-flavone and 8-methoxyfurano-(4,5-6,7)-flavone isolated from leaves(Ind j chem. 1976,14B,229; ibid .1977,15b,536)
Pharmacology: Pongapipnone A inhibited interleukin-1 production (Chem. Pharm. Bull. 1992, 40,2041) Pongaemia pinnata, which are been used as a useful treatment of diseases like diarrhoea, flatulence and for cleaning ulcer and sores. It is considered to be helpful in piles, a paste of seeds mixed with butter being used in bleeding piles. The juice of leaves is used to treat flatulence, dyspepsia, diarrhoea and cough; it is considered a remedy for leprosy and gonorrhoea. A hot infusion of the leaves is used as a medicated bath to relieve rheumatic pains and for cleaning ulcers and sores. A The juice of the roots is also used for cleaning sores, as well as for cleaning teeth and strengthening gums; the stem bark is used to treat bleeding piles.
   Moringa olifera Family: Moringaceae
Botanical description: Indigenous to northwestern India and Pakistan and plentiful on recent alluvial land or near riverbeds and streams. Cultivated throughout india primarily for its abundant, edible pods; it thrives in tropical climate of southern India. A small or medium-sized tree upto 10 m tall, with thick, soft, corky, deeply fissured bark and tomentose twigs; roots pungent. Leaves usually tripinnate, to 45 cm long; pinnae and pinnules opposite, decidous; leaflets 1.2-2 cm long and 0.6-1 cm wide, the lateral elliptic, the terminal obovate. Flowers white, fragrant, in large panicles. Fruit (pods) pendulous, green, 22-50 cm or more in length, triangular, 9 ribbed; seeds trigonous, the wings angled. Flowers and fruits once or twice each year, depending on locality; in central India, where trees remain leafless between December-January-February, flowering occurs mainly between November and march, and fruiting from February to June.
Medicinal uses: Nearly all parts of the tree are used in traditional Indian medicine, the roots, leaves and seeds being of particular importance in Ayurveda. The root and root bark are considered carminative, stomachic and abortifacient; they are applied externally to cure inflammation swellings. An infusion of the roots is used in Ayurveda to treat asthma, gout, rheumatism and inflammation; the fresh roots are used as a stimulant to treat paralytic affections, epilepsy, intermittent fever, and as a cardiac and circulatory tonic. In the form of compound spirit, they are used to treat fainting, giddiness, nervous debility, spasmodic affections of the bowels, flatulence. The leaves are rich in vitamin A and C and are considered useful in the treatment of scurvy and catarrhal affections; they are also used as a galactagogue. The crushed leaves are taken in the form of tablets to relieve stomach pain during mensturation by women in northeastern Karnataka. A paste of leaves is applied externally to promote healing of wounds. The juice extracted from leaves has antibacterial and antimalarial properties. The flowers are used as a tonic, aphrodisiac, and diuretic. Both the flower and roots contain pterygospermin, an antibiotic that is highly effective in treatment of cholera. The fruit (pod) is used to treat diseases of liver and spleen, articular pains, tetanus and paralysis. The seeds are considered as antipyretic and sometimes used to treat venereal affections.
Phytochemistry: 4-(α-L Rhamnosyloxy benzylisothiocynate and 4-(α-L-rhamnosyloxy)phenylacetonitrile isolated from raw seeds (Philipp. J. Sci 1990,119,23; Chem Abstr 1992,116,262345a); mucilage named drumstick polysaccride (DSP), isolated from pods consisted of galactose, dextrose, xylose, and sodium, potassium, magnesium, and calcium salts of glucoronic acid (Ind J Pharm Sci, 1992,54,28), new hypotensive thiocarbamates-niazinin A, niazinin B, niaziminins A& B-isolated from leaves and characterized (J. Chem. Soc. Perkin, 1992, 3237).
Pharmacology: 4-(α-L Rhamnosyloxy benzylisothiocynate exhibited antibacterial activity against Bacillus Substilis. All parts of the tree are used in traditional Indian medicine, the roots, leaves and seeds being of particular importance in pharmacological action. In the form of compound spirit, they are used to treat fainting, giddiness, nervous debility, spasmodic affections of the bowels, flatulence. The roots are used as a pungent-tasting condiment or garnish in the same way as those of true horse radish (Armoracia rusticana:Brassicaceae); the root bark, rich in alkaloids (notably moringine, a toxic substance allied to ephedrine), must be completely removed before it is used for this purpose.
   Cassia occidentalis Family: caesalpiniaceae
Botanical description: Pantropical in distribution, and found throughout India as a weed in waste places and near dwellings from the Himalayas to cape comorin, up to 1500 m elevation in northern India. An erect, fetid, woody herb or undershrub, 60-150 cm tall, with a smooth, purplish or green stem. leaves alternate, pinnate, 15-20 cm long, stipulate with a sessile dark brown gland near the base of the petiole; leaflets 3-5 pairs, opposite, short-stalked, membranous, ovateor lanceolate, 3-9 cm long and 1.5-4 cm wide, base rounded, apex acute or attenuate, glabrousabove, glaucous beneath. Flowers yellow, in short, few flowered axillary or terminal racemes; pedicels spreading, 5 mm long, elongating to 1.3 cm in fruit; bracts 1.3 cm long, ovate, acuminate, caduceus, white tinged with pink; calyx 1 cm long, divided to the base, glabrous, segments 5, white or creamy-yellow tinged with pink, oblong, obtuse membraneous; petals 5, equal, 1.3 cm long, ovate-oblong, obtuse, yellow with faint orange veins. Fruits (pods) recurved, glabrous, compressed, 10-13 cm long, and 0.8 cm wide, containing numerous dark olive green, ovoid, compressed, smooth, hard, shiny seeds 6 mm long and 4 mm wide. Flowers and fruits throughout the year depending on locality, but mainly between august and January in central India.
Medicinal uses: All parts of the plant have similar medicinal properties, and are considered purgative, tonic, febrifungal, expectorant and diuretic. The plant is used as a vermifuge and to treat sore eyes, haematuria, rheumatism, thyphoid, asthma, blood disorders, an is reportedly effective against leprosy. A decoction of plants is used for the treatment of hysteria, dysentery and other stomach trouble, and as an external application for relieving sores, rectal inflammation. It forms part of a patented herbal drug (Liv-52) that is effective in treating early cases of hepatic cirrhosis having steatorrhoea. The volatile oil obtained from leaves, roots, and seeds has antibacterial and antifungal properties. The leaves have purgative, febrifungal, tonic, diuretic, and stomachic properties; they are used to treat cough and hysteria. the leaves are also used externally to relieve inflammatory swelling, rheumatism, wounds and sprains, and are also given in the treatment of jaundice headache and toothache.
Phytochemistry: Roots contain free (1.9%) and total (4.5%) anthraquinones, a phytosterol, 1,8-dihydroxyanthraquinone, an anthraquinone, mp 179°, a hydroxyanthraquinone, mp128°, emidin, quercitin, and a substance similar to rhein isolated (Anais fac farn proto 1964,24,65; Chem abstr, 1965,63,17787b) diahthronic heteroside from leaves; C-flavonosides of apigenin from pericarp; Chrysophanol and emodol from young roots (Ann. Pharm. Fr. 1968,26,673; Chem abstr, 1969,70,84918m). matteucinol-7-rhamnoside and jaceidin-7-rhamnoside from leaves (Phytochemistry 1977,16,1107), Chrysophanol nad a bianthraquinone III isolated from leaves (Planta med 1977,32,375).
Pharmacology: seeds are used for winter cough as a cure for convulsion in children and as an excellent diuretic (experentia 1973,29,141). A decoction of plants is used for the treatment of hysteria, dysentery and other stomach trouble, and as an external application for relieving sores, rectal inflammation. It forms part of a patented herbal drug (Liv-52) that is effective in treating early cases of hepatic cirrhosis having steatorrhoea. The roots bark is used as a quinine substitute to cure fever, and its decoction is used as a stimulant and as a specific medicine for gonorrhoea and hepatic troubles. The seeds are bitter and have tonic, they are considered a blood tonic and excellent diuretic, and are also used to treat cough, whooping cough,convulsion and certain heart diseases.
   Albizzia lebbeck Family: mimosaceae
Botanical description: Probably native to tropical mainland Asia from Pakistan to Myanmar but widely introduced as a garden or roadside ornamental and naturalized in many tropical and northern subtropical countries worldwide. It is common throughout India, from the plains upto 900 m elevations in the Himalayas. It is a large, unarmed, decidous tree to about 20 m tall with a spreading crown; bark pale;young shoots glabrous. Leaves bipinnate; rachis with a gland near the base of the petiole and one below uppermost pairs of pinnae; pinnae usually 2-3 pairs, 7-12 cm long; leaflets 5-9 pairs, 2.5-4.5 cm long and 1.6-2 cm wide, with glands between their bases,elliptic-oblong or obovate-oblong, unequal-sided, retuse or obtuse at apex, base obliquely rounded or truncate; petiolules very short. Flowers fragrant, white to greenish-yellow, borne in globose umbellate heads 2-3.8 cm in diameter; peduncles 3.8-7.5 cm long, solitary or 2-4 together from the axils of the upper leaves, pedicels 2.5-3 mm long; calyx 4 mm long, pubescent, teeth short, deltoid; corolla 1 cm long, tube glabrous, lobes2.5 mm long, triangular, acute, pubescent outside; stamens longer than the corolla. Fruits (pods) 10-30 cm long and 24.5 cm wide, linear-oblong, bluntly pointed, thin green turning straw-coloured on maturity, reticulately veined above the seeds, smooth, shining; seeds 4-12, pale brown, ellipsoid-oblong, compressed. Flowers from April to June and fruits mature in December in northern and central India; in southern India flowering occurs earlier, from January to April. In central India trees remains leafless for a month or more between March and June.
Medicinal uses: In Ayurveda the astringent root is used to treat hemicrania; the acrid bark is reportedly used to treat diseases of the blood, leucoderma, itching, skin diseases, piles, inflammation, and bronchitis; the leaves are used to treat opthalmia, and the flowers for asthma. In unani medicine the root is used to treat ophthalmia; the bark is regarded as anthelmintic and used to relieve toothache and to strengthens the gums and teeth, and to treat leprosy, deafness, boils, scabies, syphilis and paralysis; the leaves are reportedly useful for treating night blindness; and the seeds are used to treat gonorrhoea and tuberculous glands, their oil applied locally for leucoderma. The flower are used as a cooling medicine and as an external application to relieve boils, skin eruption and swelling. the seed oil is used externally to promote healing of lesions in leprosy among the Irulars of Tamilnadu.
Phytochemistry: A new acyclic ester isolated and characterized as heneicos-7 (2)-enyl-24-hydroxytetracos-10 (2)-enoate; lupeol, oleanolic acid, docosanoic acid and β-sitosterol also was isolated (Ind J Pharm Sci, 1991,53,24). Echinocystic acid β-sitosterol identified in bark and seeds (Ind J Appl. Chem, 1969,32,73; Chem Abstr 1971,75,160352). Mature leaves contained ketoacids including phosphoenol pyruvate, glyoxalate, oxaloacetate and α-oxoglutarate (Plant Biochem J, 1977,4,34; Chem Abstr 1977,17,148762s); vicenin-2, reynoutrin, rutin, myricitrin and robinin from leaves (shoyakugaku Zasshi 1977,31,172; Chem Abstr 1978,88,14897h).
Pharmacology: Bark and flowers decoction protected guinea pig against histamine and acetlycholine-induced bronchospam. Chronic treatment with bark decoction also protected sensitized guinea pigs against antigen challenge. Drug showed anti-asthmatic and anti-anaphylactic activities due to inhibition of phenomenon of sensitization (Ind J Pharmacol, 1977,9,189). Saponin showed β-haemolysis against buffalo and sheep blood and α-haemolysis against human blood. Purified saponin showed antifungal activity against marcophomina phaseolina (mic 32.8 μg), stemphilum species and fusarium solani (Pak Vet J 1990,10,146; Chem Abstr 1992,116,102686j). The plant has been showed to poses antidirrhoeal and antifertility activities. It posses anti anaphylactic and anticonvulsive properties. The leaves are reportedly useful for treating night blindness; and the seeds are used to treat gonorrhoea and tuberculous glands, their oil applied locally for leucoderma.

The present invention provides a novel herbal formulation(s) obtained from decoction of four potent plants having the property of improving the general health and used in treatment of diseases of blood, stomach problem and piles. Novelty of the invention resides in herbal formulation having the property nerves relaxant properties and antinociceptive properties and used as intoxicant, stomachic, anti-bacterial, expectorant, tonic, rheumatism, boil and swelling.

The herbal anti-hemorrhoids synergistic formulation (s) contains extracts of plants in pharmacologically effective form. The plants are selected from Pongaemia pinnata, Moringa olifera, Cassia occidentalis and Albizia lebbeck and are useful in the treatment of piles and hemorrhoids. The composition is a synergistic mixture of plant extracts having anti-inflammatory, cooling, diuretic, nerves relaxant properties and antinociceptive properties.

The amounts of the extracts are as follows: Moringa olifera (2-4 Wt %), Pongaemia pinnata (3-4 Wt %), Cassia occidentalis (2-4 Wt %) and Albizia lebbeck (1-4 Wt %) and balance being conventional additives. The extract of Moringa olifera is obtained from leaves. The extract of Pongaemia pinnata is obtained from leaves. The extract of Cassia occidentalis is obtained from whole plant and the extract of Albizia lebbeck is obtained from bark.

The formulation also has the property of intoxicant and antibacterial, antifungal and used in treatment of diseases of blood, stomach problem and piles. The formulation is also used to ulcer and sores and in relieving rectal inflammation. The formulation is also used to cure wounds and fractures. The herbal formulation shrinks piles mass, control bleeding and hastens the healing process in inflamed skin and mucous membranes. The formulation has anti-inflammation properties and reduces constipation, which is one of the important criteria for being an effective measure against haemorrhoids. The relief from pain and and soothing action facilitates the smooth evacuation of faeces. The laxative property corrects chronic constipation associated with haemorrhoids.

As a result of intensive study conducted by the inventors with the aim of achieving aforementioned objectives, new herbal formulation(s) has been obtained to ameliorate the symptoms of disease and to improve the general health of the patient. It was shown that it produces a significant improvement in condition of plies and its treatment for the same.

The invention thus meets the need for a new process in which the optimal proportions of vitamins, amino acids, long chain fatty acids and active therapeutic marker compounds are retained in the product and underlies the efficacy of the compound as an effective in the form of capsule or ointment for treatment of piles.

The inventive formulation is also suitable for encapsulation in gelatin shells to form soft gels/ointments/capsules. Regardless of the particular form in which the inventive formulation is prepared, the daily dosage of it to experimental animals fall within the ranges set forth above. Depending on the concentration of the inventive formulation composition in the above form, the total amount of the food product per serving or encapsulated capsule etc will also vary the desired therapeutic activity.

The invention is further illustrated by the following non-limiting examples.

Formulation (F1)

| Moringa olifera | 2 | wt. % |
|---|---|---|
| Albizia lebbeck. | 4 | wt. % |
| Pongaemia pinnata | 4 | wt. % |
| Sucrose/Lactose | 66.7 g/1.2 g | |
| Alcohol | 10 | wt. % |

| | |
|---|---|
| Water | q.s. to make 100 ml |

Formulation (F2)

| Cassia occidentalis | 4 | wt. % |
|---|---|---|
| Albizia lebbeck. | 4 | wt. % |
| Pongaemia pinnata | 4 | wt. % |
| Sucrose/Lactose | 66.7 g/1.2g | |
| Alcohol | 10 | wt. % |

| | |
|---|---|
| Water | q.s. to make 100 ml |

Formulation (F3)

| Albizia lebbeck. | 4 | wt. % |
|---|---|---|
| Pongaemia pinnata | 4 | wt. % |
| Sucrose/Lactose | 66.7 g/1.2 g | |
| Alcohol | 10 | wt. % |

| | |
|---|---|
| Water | q.s. to make 100 ml |

Formulation (F4)

| Pongaemia pinnata | 4 | wt. % |
|---|---|---|
| Moringa olifera | 4 | wt. % |
| Cassia occidentalis | 4 | wt. % |
| Albizia lebbeck. | 4 | wt. % |
| Sucrose/Lactose | 66.7 g/1.2 g | |
| Alcohol | 10 | wt. % |

| | |
|---|---|
| Water | q.s. to make 100 ml |

Pongaemia pinnata, Moringa olifera, Cassia occidentalis, Albizia lebbeck, were collected and dried in shade. The dried material (1 Kg) is then powdered and extracted with 50% aqueous alcohol (3 L) for 5 days. At the end of this, the solvent is decanted and filtered if necessary to remove the plant debris. The extract is then concentrated under vacuum at less than 50° C., and then the extract is lyophilised to obtain the extract in powder form.

Mix the plant extracts and dissolve them in 500 ml 10% alcohol, filter the solution and make up the volume with required amount of water to make 100 ml.

The formulation is useful to as a capsule/ointment and treating piles. Accordingly, the investigation deals with the oral dosage form has been described in detail giving the formula of the ingredients along with the method and mode of usage of the standardized herbal formulation. Kindly refer table I and II.

TABLE 1
Effect of Formulations (F1-F4) on λ carrageenin induced
edema in rats.

| Treatment | Dose | Paw volume (ml) at 3h | |
|---|---|---|---|
| (mg/kg) | (mg/kg) | λ carrageenin | % Inhibition |
| Control | — | 0.98 ± 0.01 | — |
| Formulation F1 | (200 mg/kg) | 0.84 ± 0.02 | 14.28 |
| Formulation F2 | (200 mg/kg) | 0.69 ± 0.04 | 29.59 |
| Formulation F3 | (200 mg/kg) | 0.41 ± 0.04 | 58.18^{a} |
| Formulation F4 | (200 mg/kg) | 0.21 ± 0.04 | 78.57^{b} |
| Indomethacin | (100 mg/kg) | 0.15 ± 0.02 | 84.69^{b} |

Values are mean ± SEM for six rats.
P: ^{a}<0.01 and ^{b}<0.001 compared to control group.

Formulation F1 contains Pongaemia pinnata, Moringa olifera and Albizia lebbeck. Formulation F2 contains Pongaemia pinnata, Cassia occidentalis and Albizia lebbeck. Formulation F3 contains Pongaemia pinnata and Albizia lebbeck. Formulation F4 contains Pongaemia pinnata, Moringa olifera, Cassia occidentalis and Albizia lebbeck.

Antiinflammatory Activity

Carrageenin induced paw edema: Rats were injected with 0.1 ml of 1% λ carrageenin (St.-Louis, Mo.) into the subplantar side of the left hind paw. The paw was marked with ink at the level of the lateral malleolus and dipped in perspex cell up to this mark. The paw volume was measured with an Ugo Basile Plethysmometer (No: 61402) (7140 Comerio-varese, Italy) immediately and 3 h after injecting the λ carrageenin suspension. The alcoholic root extracts of Formulation (F1-F4) was administered at dose of 200 mg/kg respectively orally by gavage 1 h before the λ carrageenin injection. Significant reductions in the paw volume compared to vehicle treated control animals were considered as anti-inflammatory response. Percentage inhibition of oedema was calculated as follows:
% Inhibition=(1−V_{T}/V_{C})×100

V_{T}=Paw volume in drug treated rats, V_{C}=Paw volume in control group of rats.

TABLE 2
Effect of Formulations (F1-F4) on force-induced pain in rats.

| | | Weight causing pain (g) | |
|---|---|---|---|
| Treatment | Dose | Before | After |
| (mg/kg) | (mg/kg) | Administration | Administration |
| Formulation F1 | (200 mg/kg) | 79.4 ± 5.2 | 82.6 ± 5.2 |
| Formulation F2 | (200 mg/kg) | 86.5 ± 5.8 | 98.7 ± 5.3 |
| Formulation F3 | (200 mg/kg) | 85.9 ± 6.1 | 125.6 ± 7.0^{a} |
| Formulation F4 | (200 mg/kg) | 88.4 ± 5.5 | 149.2 ± 6.5^{b} |
| Ibuprofen | (100 mg/kg) | 84.7 ± 5.7 | 161.1 ± 7.9^{b} |

Values are mean ± SEM for six rats.
P: ^{a}<0.01 and ^{b}<0.001 compared to control group.

Formulation F1 contains Pongaemia pinnata, Moringa olifera and Albizia lebbeck. Formulation F2 contains Pongaemia pinnata, Cassia occidentalis and Albizia lebbeck. Formulation F3 contains Pongaemia pinnata and Albizia lebbeck. Formulation F4 contains Pongaemia pinnata, Moringa olifera, Cassia occidentalis and Albizia lebbeck.

Antinociceptive Activity

Analgesy-meter induced pain: The analgesic effect of the powder was tested in rat of either sex, using an Ugo Basile Analgesy meter (No. 32725) (21025 Comerio-varese, Italy). This method involves the application of force to the paw of the rat using the Analgesy-meter, which exerts a force that increases at a constant rate. The rat was gently placed between the plinth and plunger. The instrument was switched on and a constant motor rate was used to drive the plunger on to the paw of the rat. When the rat struggles, the instrument is switched off and the force at which the animal felt pain was read on a scale calibrated in grams ×10 by a pointers. The pre-treatment and the after treatment weight causing pain was determined for each rat. The alcoholic root extract of Formulation (F1-F4) was administered at a dose of 200 mg/kg respectively 30 min before testing.

TABLE 3
Age-Sex incidence and incidence of the symptoms of piles

| | No of | | |
|---|---|---|---|
| Age in years | patients | Male | Female |
| 15-20 yrs. | 4 | 4 | — |
| 20-25 yrs | 6 | 5 | 1 |
| 25-30 yrs | 10 | 8 | 2 |
| 30-35 yrs | 10 | 3 | 7 |
| Total cases | 30 | 20 | 10 |

Thirty cases of piles were treated, of which 20 were males and 10 were females. The greatest incidence of symptoms was found between age 20 and 30. This is the period of greatest physical activity.

Clinical Study

Thirty patients with bleeding per rectum who had piles; mainly first and second degree and a few third degrees were selected for the present study. Piles due to some other causes (malignancy, other systemic diseases etc) were excluded from the purview of the present study. Patients were grouped into the following three degrees:

1^{st} degree—congested blood vessels, but no prolapse.

2^{nd} degree—prolapsed on straining but regressed spontaneously.

3^{rd} degree—continuously prolapsed

A details history including the age, sex occupation, diet, family history, bowel habits, duration of bleeding, pain in the perineum, discharge, itching together with other general and local symptoms were noted.

In every case, general systemic examination proctoscopic and digital per rectal examinations was performed. In all cases, the number, size, situation, colour and degree of piles were noted diagrammatically. Routine stool, urine, blood examinations were done. All patients were subjected to Formulation (F4) containing Pongaemia pinnata, Moringa olifera, Cassia occidentalis and Albizia lebbeck.

Schedule(s):

1. Duration of the course: 6 weeks
2. Dose (200 mg): Formulation (F4) thrice daily for 1 week or till the alleviation of symptoms (usually 2-3 weeks) followed by 1 capsule thrice daily for the rest of the course. Formulation (F4) in the form of ointment applied per rectal at least thrice a day.
3. Follow up—a) Every week for six weeks
   b) Then, fortnightly for 3 months
   c) Then, monthly for rest of trial period (upto 1 year) or as deemed necessary.

Observations: Out of 30 cases studied, 15 cases had first-degree piles, 12 cases had second-degree piles and 3 had third degree piles. There were 20 males and 10 females. None of the females had any symptoms associated with pregnancy or childbirth. All 30 cases were non-vegetarian. The bleeding had no definite relation with occupation; as amongst males, 6 were computer operator, 10 were manual labourer, 4 were students. All females were housewives except 4 were unmarried and were bank employee also. All had suffered from intestinal amoebiasis, at least once in their lifetime. All females and 20 males were constipated. All constipated patients used to take some laxatives in the form of drug or diet.

TABLE 4
Effect of formulation F4 in 1^{st} degree of piles (1^{st} degree symptoms are-
congested blood vessels, but no prolapse)

| | No of | Complete | | No |
|---|---|---|---|---|
| Signs & symptoms | cases | relief | Reduced | change |
| 1. Bleeding per rectum | 7 | 3 | 2 | 2 |
| Occasional blood & mucous | 3 | 1 | 1 | 1 |
| in stool | | | | |
| Bleeding without pain | 4 | 2 | 1 | 1 |
| Bleeding with pain | — | — | — | — |
| 2. Constipation | 9 | 3 | 4 | 2 |
| 3. Perianal itching | 6 | 2 | 3 | 1 |
| 4. Prolapsed piles | — | — | — | — |
| (continuously) | | | | |
| 5. Prolapsed during | — | — | — | — |
| defecation but regressed | | | | |
| spontaneously | | | | |

Formulation F4 contains Pongaemia pinnata, Moringa olifera, Cassia occidentalis and Albizia lebbeck.

Out of 7 cases 3 patients showed positive results (complete relief), 2 cases (Reduced) and 2 cases showed no change. Where as 4 cases reduced the constipation when treated with formulation F4.

TABLE 5
Effect of Formuation F4 in 2^{nd} degree of piles (2^{nd} degree-prolapsed on
straining but regressed spontaneously)

| Signs & symptoms | No of cases | Complete relief | Reduced | No change |
|---|---|---|---|---|
| 1. Bleeding per rectum | 12 | 9 | 2 | 1 |
| Occasional blood & mucous in | 6 | 4 | 1 | 1 |
| stool | | | | |
| Bleeding without pain | 2 | — | 1 | 1 |
| Bleeding with pain | 4 | 2 | 1 | 1 |
| 2. Constipation | 13 | 9 | 2 | 2 |
| 3. Perianal itching | 10 | 6 | 2 | 2 |
| 4. Prolapsed piles | 6 | 3 | 2 | 1 |
| (continuously) | | | | |
| 5. Prolapsed during | 8 | 4 | 2 | 2 |
| defaecation but regressed | | | | |
| spontaneously | | | | |

Formulation F4 contains Pongaemia pinnata, Moringa olifera, Cassia occidentalis and Albizia lebbeck.

TABLE 6
Effect of formulation F4 in 3^{rd} degree of piles (3^{rd} degree-continuously
prolapsed)

| Signs & symptoms | No of cases | Complete relief | Reduced | No change |
|---|---|---|---|---|
| 1. Bleeding per rectum | 7 | 6 | 1 | — |
| Occasional blood & mucous in | 5 | 4 | 1 | — |
| stool | | | | |
| Bleeding without pain | 1 | 1 | — | — |
| Bleeding with pain | 1 | 1 | — | — |
| 2. Constipation | 8 | 7 | 1 | — |
| 3. Perianal itching | 7 | 6 | 1 | — |
| 4. Prolapsed piles | 8 | 6 | 2 | — |
| (continuously) | | | | |
| 5. Prolapsed during | 8 | 5 | 3 | — |
| defaecation but regressed | | | | |
| spontaneously | | | | |

Formulation F4 contains Pongaemia pinnata, Moringa olifera, Cassia occidentalis and Albizia lebbeck.

In the first-degree hemorrhoids the response to herbal formulation therapy was very good. Many patients were completely free from bleeding which is shown in table 4. The other symptoms were also showed significant reduction with the treatment. In second-degree hemorrhoids the response to therapy was also good. In third degree the formulation therapy show excellent results. There was the disappearance of some symptoms from all the degree of piles. The above that there was no toxic or side reaction in any cases. Based on our clinical trial we believe herbal formulation therapy is very satisfactory for the conservative treatment of piles, in first and second-degree piles. In third degree hemorrhoids herbal formulation therapy gives considerable relief from symptoms. Styplon is also found as a haemostatic drug in conjunction with herbal formulation therapy. From the above table and result it showed that the herbal formulation is very effective against the treatment of piles.

We claim:

1. A herbal synergistic formulation comprising extracts of Moringa olifera, Pongaemia pinnata, Cassia occidentalis and Albizia lebbeck in pharmaceutically acceptable dosages optionally along with an additive useful in treatment of piles and hemorrhoids.

2. Herbal formulation as claimed in claim 1, wherein the extracts/juice of the plants are mixed in the ratio consisting of Moringa olifera 2-4 Wt %, Pongaemia pinnata 3-4 Wt %, Cassia occidentalis 2-4 Wt % and Albizia lebbeck 1-4 Wt % the balance being conventional additives.

3. Herbal formulation as claimed in claim 1 wherein the extract of Moringa olifera is obtained from leaves.

4. Herbal formulation as claimed in claim 1 wherein the extract of Pongaemia pinnata is obtained from leaves.

5. Herbal formulation as claimed in claim 1 wherein the extract of Cassia occidentalis is obtained from whole plant.

6. Herbal formulation as claimed in claim 1 wherein the extract of Albizia lebbeck is obtained from bark.

7. Herbal formulation as claimed in claim 1 wherein the additives added are selected from the group consisting of sugar, alcohol and water quantity sufficient to make 100%, for converting into a syrup form.

8. Herbal formulation as claimed in claim 1, wherein the formulation at a dose range of 100-200 mg of formulations for 1 week or till alleviation of symptoms within 2-3 weeks showed significant anti-inflammatory and analgesic activity in the rectal region.

9. Method for the treatment of piles and hemorrhoids comprising administering to a subject a pharmaceutically effective amount of a herbal formulation consisting of extracts of Moringa olifera, Pongaemia pinnata, Cassia occidentalis and Albizia lebbeck in pharmaceutically acceptable dosages optionally along with an additive.

10. Method as claimed in claim 9 wherein the formulation is anti-inflammatory.

11. Method as claimed in claim 9 wherein the formulation relieves rectal inflammation and treats ulcer conditions associated with piles.

12. Method as claimed in claim 9 wherein the formulation shrinks piles mass, controls bleeding and enhances healing process in inflamed skin and mucous membranes.

13. Method as claimed in claim 9 wherein the formulation has anti-inflammatory activity and reduces constipation.

14. Method as claimed in claim 9 wherein the formulation relieves pain and soothes the subject thereby facilitating smooth evacuation of faeces.

15. Method as claimed in claim 9 wherein the formulation acts as a laxative.

16. Method as claimed in claim 9 wherein the subject is a human being.

17. Method as claimed in claim 9 wherein the formulation is used at a dose range of 100-200 mg of formulations for 1 week or till alleviation of symptoms within 2-3 weeks.

18. Use of a herbal formulation for treatment of piles and hemorrhoids the formulation comprising pharmaceutically effective amount of a herbal formulation consisting of extracts of Moringa olifera, Pongaemia pinnata, Cassia occidentalis and Albizia lebbeck in pharmaceutically acceptable dosages optionally along with an additive.

19. Use as claimed in claim 18 wherein the formulation is anti-inflammatory.

20. Use as claimed in claim 18 wherein formulation relieves rectal inflammation and treats ulcer conditions associated with piles.

21. Use as claimed in claim 18 wherein the formulation shrinks piles mass, controls bleeding and enhances healing process in inflamed skin and mucous membranes.

22. Use as claimed in claim 18 wherein the formulation has anti-inflammatory activity and reduces constipation.

23. Use as claimed in claim 18 wherein the formulation relieves pain and soothes the subject thereby facilitating smooth evacuation of faeces.

24. Method as claimed in claim 9 wherein the formulation acts as a laxative.

25. Method as claimed in claim 9 wherein the subject is a human being.

26. Method as claimed in claim 9 wherein the formulation is used at a dose range of 100-200 mg of formulations for 1 week or till alleviation of symptoms within 2-3 weeks.
